(19) United States
(12) Patent Application Publication
(10) Pub. No.: US 2005/0260229 A1
(43) Pub. Date: Nov. 24, 2005

(54) Glycans of Anaplasma marginale major surface protein 1a, pharmaceutical compositions and methods of use

(76) Inventors: Jose de Jesus de la Fuente, Stillwater, OK (US); Katherine M. Kocan, Perkins, OK (US); Edmour F. Blouin, Perkins, OK (US); Jose Carlos Garcia-Garcia, Baltimore, MD (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/089,601

(22) Filed: Mar. 25, 2005

Related U.S. Application Data
(63) Continuation-in-part of application No. 10/285,319, filed on Oct. 31, 2002.
Application No. 10/285,319 is a continuation-in-part of application No. 10/002,636, filed on Oct. 26, 2001, now Pat. No. 6,939,549.
(60) Provisional application No. 60/244,333, filed on Oct. 30, 2000.

(51) Int. Cl. A61K 39/005; C07K 14/195

(52) U.S. Cl. 424/234.1; 530/395

(57) ABSTRACT

Glycans derived from the N-terminal repeated peptides of Anaplasma marginale major surface protein (MSP)1a are characterized. Pharmaceutical compositions including such epitopes, and methods useful to induce or enhance an immune response against infection by ehrlichial parasites of the species Anaplasma marginale, are provided.

### CROSS REFERENCE TO RELATED APPLICATION

This application is a continuation-in part of prior filed, copending U.S. patent application Ser. No. 10/285,319, filed Oct. 31, 2002, which is a continuation-in-part of U.S. patent application Ser. No. 10/002,636, filed Oct. 26, 2001, which claims the benefit of U.S. provisional patent application Ser. No. 60/244,333, filed Oct. 30, 2000, all of which are hereby incorporated by reference.

### BACKGROUND OF THE INVENTION

1. Technical Field

The present invention relates to glycans of Anaplasma marginale major surface protein (MSP)1a, related pharmaceutical compositions and methods useful to induce or enhance an immune response against infection by ehrlichial parasites of the species Anaplasma marginale.

2. Background

Anaplasmosis is a tick-borne disease of cattle caused by the obligate intraerythrocytic rickettsia Anaplasma marginale (Rickettsiales: Anaplasmataceae). The acute phase of the disease is characterized by severe anemia, weight loss, fever, abortion, lower milk production and often death (28). The only known site of infection of A. marginale in cattle is within erythrocytes (41). The number of infected erythrocytes increases geometrically and removal of these infected cells by phagocytosis results in development of anemia and icterus without hemoglobinemia and hemoglobinuria. Cattle that recover from acute infection remain persistently infected, are protected from clinical disease, and serve as reservoirs of A. marginale for mechanical transmission and for biological transmission by ticks (21, 23).

The process of infection of host cells by A. marginale is initiated by adhesion of the rickettsia to the host cell membrane (7), a process that appears to be mediated by surface-exposed proteins on the pathogen and host cell receptors. Of the five major surface proteins (MSPs) identified on erythrocytic and tick stages of A. narginale, the MSP1 complex, composed of two polypeptides, MSP1a and MSP1b, has been shown to be involved in adhesion of A. marginale to host cells (3, 15, 32). Immunization of cattle with the MSP1 complex has also been shown to induce partial protective immunity (37).

The A. marginale MSP1a is encoded by a single gene, msp1a (1), while MSP1b is encoded by at least two genes, msp1β1 and msp1β2 (4, 10, 13). MSP1a has been shown to contain a neutralization-sensitive epitope (39) and to be an adhesin for both bovine erythrocytes and tick cells, whereas MSP1b is an adhesin only for bovine erythrocytes (3, 15, 32). The extracellular N-terminal region of MSP1a contains tandemly repeated peptides (17, 18) which have been shown to be necessary and sufficient for adhesion of A. marginale to tick cells and bovine erythrocytes (17). MSP1a has also been shown to be involved in infection and transmission of A. marginale by Dermacentor spp. (8, 16).

The molecular mass of both MSP1a and MSP1b has been found to be greater than the molecular weight predicted from their respective amino acid sequences (1, 3, 36). Surface proteins of other rickettsial organisms, specifically Ehrlichia chaffeensis P120 and E. canis P140, were shown to be glycosylated, which accounted for the difference between their expected and observed molecular masses (30). In addition, surface proteins from other Gram-negative bacteria have been shown to be glycosylated and the glycosylation appears to be involved in their ability to adhere to and invade host cells (5). In connection with the present invention, it was discovered that MSP1a and MSP1b from A. marginale are glycosylated. The glycosylation of the native and recombinant MSP1a and MSP1b proteins was characterized and role of these carbohydrate moieties in the adhesive properties of MSP1a for tick cells was studied.

### SUMMARY OF THE INVENTION

Anaplasma marginale, the causative agent of bovine anaplasmosis, is a tick-borne rickettsial pathogen of cattle that multiplies in erythrocytes and tick cells. Major surface protein (MSP)1a and MSP1b form the MSP1 complex of A. marginale which is involved in adhesion of the pathogen to host cells. It was theorized that MSP1a and MSP1b were glycosylated because the observed molecular weights of both proteins were greater than the deduced molecular masses. It was further theorized that the glycosylation of MSP1a plays a role in adhesion of A. marginale to tick cells. Native and Escherichia coli-derived recombinant MSP1a and MSP1b proteins were shown by gas chromatography to be glycosylated and to contain neutral sugars. Glycosylation of MSP1a appeared to be mainly O-linked to Ser/Thr residues in the N-terminal repeated peptides. Glycosylation may play a role in adhesion of A. marginale to tick cells because chemical deglycosylation of MSP1a significantly reduced its adhesive properties. Although the MSP1a polypeptide backbone alone was adherent to tick cell extract, the glycans in the N-terminal repeats appeared to enhance binding and may cooperatively interact with one or more surface molecules on host cells. These results demonstrate that MSP1a and MSP1b are glycosylated and suggest that the glycosylation of MSP1a plays a role in the adhesion of A. marginale to tick cells.

In one aspect of the present invention there are thus provided novel glycans derived from MSP1a purified surface protein antigen of A. marginale.

In another aspect of the present invention there is provided a pharmaceutical composition for inducing or enhancing an immune response in a ruminant against A. marginale, the composition comprising one or more glycans derived from MSP1a purified surface protein antigen of A. marginale, alone or in combination with other antigenic components, wherein the composition further comprises a pharmaceutically acceptable carrier or diluent.

In still another aspect of the present invention there is provided a method for inducing or enhancing an immune response in a ruminant to provide immune protection against infection by A. marginale, the method comprising administering to said ruminant an effective amount of the inventive pharmaceutical composition.

A better understanding of the present invention, its several aspects, and its advantages will become apparent to those skilled in the art from the following detailed description, taken in conjunction with the attached drawings, wherein there is shown and described the preferred embodiment of the invention, simply by way of illustration of the best mode contemplated for carrying out the invention.

### BRIEF DESCRIPTION OF THE DRAWINGS

FIG. 1 is a chart illustrating the conservation of Ser/Thr residues (highlighted) in the tandem repeats encoded by Anaplasma marginale msp1a from different isolates. The amino acid positions are indicated above the sequences. The arrowhead points to the 20th amino acid, which is involved in interaction with tick cells. The neutralization-sensitive epitope recognized by monoclonal antibody ANA15D2 is indicated by the bracket. Sequences were obtained from J. de la Fuente, L. M. F. Passos, R. A. Van Den Bussche, M. F. B. Ribeiro, E. J. Facury-Filho, and K. M. Kocan, submitted for publication.

FIG. 2 reflects the predicted glycosylation sites in (A) MSP1a and (B) MSP1b from the Oklahoma isolate of A. marginale. O-glycosylation was predicted using NetOGlyc 2.0 prediction algorithm to occur in the amino acid positions in which the O-glycosylation potential (bars) is greater than the threshold (curve). Arrowheads indicate potential N-glycosylation sites (Asn-Xaa-Ser/Thr sequences).

FIG. 3 is a western blot analysis of native and recombinant (A) MSP1a and (B) MSP1b proteins from the Oklahoma isolate (lanes 1-4) and Virginia isolate (lanes 5-7) of A. marginale. Samples of recombinant proteins expressed in E. coli (lanes 1, 5), erythrocyte-derived A. marginale (lanes 2, 6), tick cell culture-derived A. marginale (lanes 3, 7) and infected tick salivary glands (lane 4) were separated by SDS-PAGE and reacted with (A) anti-MSP1a MAb ANA15D2 or (B) rabbit polyclonal anti-MSPlb serum. Arrows on the left indicate mol. wt. markers in kDa.

FIG. 4 is a graph illustrating the dependence of the MSP1a molecular mass upon the number of tandem repeats. The predicted (squares) and observed (triangles) molecular masses of recombinant MSP1a from different A. marginale isolates expressed in E. coli were calculated from the reported amino acid sequence or estimated from the electrophoretic mobility, respectively. The intercept indicates the molecular mass of the conserved C-terminal region and the slope the average molecular mass of a single repeat.

FIG. 5 reflects the analysis of MSP1a proteins from different A. marginale isolates expressed in E. coli. Recombinant E. coli cell extracts were separated by SDS-PAGE, transferred to a nitrocellulose membrane and (A) reacted with anti-MSP1a MAb ANA15D2 or (B) stained with carbohydrate-specific periodate oxidation and biotin hydrazide conjugation. Arrowheads indicate the recombinant MSP1a protein bands. Numbers on the left indicate molecular weights in kDa. Lane 1, Negative E. coli control. Lanes 2-7, protein extract of recombinant E. coli cells expressing MSP1a protein from A. marginale isolates from Virginia (lane 2), Oklahoma (lane 3), California (lane 4), St. Maries (lane 5), Texas (lane 6), and Okeechobee (lane 7).

FIG. 6 reflects the analysis of MSP1b and mutant MSP1a proteins expressed in E. coli. Proteins were purified by FLAG-affinity chromatography, separated by SDS-PAGE, transferred to a nitrocellulose membrane and (A) reacted with anti-MSP1a MAb ANA15D2 (lane 1), anti MSP1a MAb AFOR2.2F1 (lane 2, 3), or rabbit polyclonal anti-MSP1b serum (lane 4); or (B) stained specifically for carbohydrates. Lane 1, Oklahoma isolate MSP1a repeats; lane 2, MSP1a without the repeats; Oklahoma isolate MSP1a (lane 3), and MSP1b (lane 4). Arrowheads indicate recombinant protein bands. Numbers on the left indicate molecular weights in kDa.

FIG. 7 reflects the chemical deglycosylation of MSP1a with TFMS. Native (lanes 1-3) and deglycosylated (lanes 4-6) MSP1a was transferred to a nitrocellulose membrane and reacted with anti-MSP1a MAb ANA15D2 (lanes 1, 4), anti-MSP1a MAb AFOR2.2F1 (lanes 2, 5), anti-FLAG M2 MAb (lanes 3, 6).

FIG. 8 is a graph illustrating the binding of glycosylated and deglycosylated MSP1a to tick cells. Recombinant MSP1a, MSP1b and deglycosylated MSP1a were assayed in vitro for their ability to bind to tick cell proteins. Binding was expressed as the OD_{450 nm} (mean±S.D.) from three replicates. Asterisks denote statistically significant difference (P<0.05) between native and deglycosylated MSP1a determined using an ANOVA test.

### DESCRIPTION OF THE PREFERRED EMBODIMENTS

Before explaining the present invention in detail, it is important to understand that the invention is not limited in its application to the details of the embodiments and steps described herein. The invention is capable of other embodiments and of being practiced or carried out in a variety of ways. It is to be understood that the phraseology and terminology employed herein is for the purpose of description and not of limitation.

In accordance with the present invention there is provided glycans derived from MSP1a useful as antigenic components of a pharmaceutical composition. They may also be utilized in combination with other antigenic components. The invention further encompasses the vaccination of ruminants using the glycans.

The present invention will be further understood with reference to the following non-limiting description of experimental results and examples.

Materials and Methods

Anaplasma marginale Isolates

The Oklahoma isolate of A. marginale was used for the cattle immunization studies and the Virginia isolate was used for challenge exposure of immunized cattle. Both of these isolates have been shown to be tick transmissible and have been propagated in cultured tick cells in our laboratory (Munderloh et al., 1996; Blouin et al., 2000; de la Fuente et al., 2001b; 2002b).

Bovine Erythrocyte-Derived A. marginale

Two splenectomized calves (3 month old, mixed breed beef cattle) were experimentally infected with the Oklahoma or Virginia isolates of A. marginale. Calf PA479 was infected with blood stabilate (Oklahoma isolate) retrieved from liquid nitrogen that was collected from calf PA407 at 10% percent parasitized erythrocytes (PPE) (6). Calf PA433 was infected with the Virginia isolate of A. marginale by allowing Dermacentor variabilis males that acquired infection on calf PA432 (19) to feed on the calf and thus transmit A. marginale. The calves were maintained by the OSU Laboratory Animal Resources according to the Institutional Care and Use of Animals Committee guidelines. Infection of the calves was monitored by examination of stained blood smears. Bovine erythrocytes were collected from the calves at peak parasitemia (PA479, PPE 32.2%; PA433, PPE 18.9%), washed three times in phosphate-buffered saline (PBS), each time removing the buffy coat, and stored at −70° C.

Tick Cell Culture-Derived A. marginale

A. marginale was propagated in the tick cell line, IDE8 (ATCC CRL 11973), derived originally from Ixodes scapularis embryos, as described previously (6, 35). Briefly, tick cells were maintained at 31° C. in L-15 B medium, pH 7.2, supplemented with 5% heat inactivated fetal bovine serum (Sigma, USA), 10% tryptose phosphate broth (Difco, USA) and 0.1% lipoprotein concentrate (ICN, USA), and the culture medium was replaced weekly. Monolayers of IDE8 cells were inoculated with the Oklahoma or Virginia isolate of A. marginale and monitored for infection by phase contrast microscopy and examination of stained smears. Terminal cell cultures were harvested by centrifugation at approximately 10 days post-inoculation for analysis of MSP1a and MSP1b.

Infection of Ticks and Collection of Salivary Glands

Dermacentor variabilis were obtained from the Oklahoma State University, Centralized Tick Rearing Facility. Larvae and nymphs were fed on rabbits and sheep, respectively, and were then allowed to molt to the subsequent stage. Male ticks were held in a humidity chamber (90-95% RH) at 25° C. with a 14-hr photoperiod until used for these studies. Uninfected males were allowed to acquire infection with the Oklahoma isolate by feeding for seven days on the infected calf PA479 during ascending parasitemia, after which the ticks were removed and held at room temperature in humidity chamber for seven days. The ticks were then allowed to transmission feed on a sheep for seven days to allow for development of colonies of A. marginale in salivary glands, after which they were removed, the salivary glands dissected, pooled and used for analysis of A. marginale MSPs.

Cloning, Expression and Purification of Recombinant MSP1a and MSP1b

The msp1α and msp1β₁ genes of the Oklahoma isolate of A. marginale, encoding for MSP1a and MSP1b, respectively, were cloned by PCR, fused to the FLAG peptide and expressed in E. coli as reported previously (15). E. coli cells expressing recombinant MSP1a and MSP1b proteins were collected and disrupted by sonication in 0.1% Triton X-100 in Tris-buffered saline (TBS). The recombinant proteins were purified by FLAG-affinity chromatography (Sigma, USA) following the manufacturer's instructions. Expression and purification of the recombinant proteins was confirmed by SDS-polyacrylamide gel electrophoresis (29) and immunoblotting.

The msp1α genes from A. marginale isolates from California, Saint Maries (Id.), Texas, Virginia and Okeechobee (Fla.) were also cloned and expressed in E. coli as described previously for the Oklahoma isolate (17).

Construction of msp1α mutants

Two msp1α mutants were constructed for expression in E. coli. The first mutant contained only the sequence encoding for the N-terminal region of the MSP1a protein that includes the tandem repeats. The second mutant contained the sequence encoding for the conserved C-terminal region of MSP1a which lacks the tandemly repeated peptides. These mutants were obtained by PCR using the Oklahoma isolate msp1α gene as described previously (17).

SDS-Polyacrylamide Gel Electrophoresis and Immunoblotting

Protein samples were loaded on 10% polyacrylamide gels that were stained with Coomassie Brilliant Blue or transferred to nitrocellulose membranes. Membranes were blocked with 5% skim milk for 60 min at room temperature. Western blot analysis was performed using monoclonal antibodies ANA15D2 (VMRD, USA) and AFOR2.2F1 (produced in our laboratory), specific for the repeats and the conserved C-terminal region of MSP1a, respectively, anti-FLAG M2 monoclonal antibody for detection of recombinant fusion proteins or MSP1b-monospecific rabbit serum for detection of MSP1b. After washing with TBS, membranes were incubated with 1:10,000 goat anti-mouse IgG or goat anti-rabbit IgG alkaline phosphatase conjugate (KPL, USA). Membranes were washed again and the color developed using BCIP/NBT alkaline phosphatase substrate (Sigma, USA).

Identification of Glycoproteins

Protein glycosylation was detected on blots of pure proteins or crude extracts by a modification of the method of Haselbeck and Hösel (9, 25). Briefly, 10 μg total protein of crude extracts or 2 μg of purified protein was loaded, separated in a 10% SDS-PAGE gel, and transferred to a nitrocellulose membrane. The membrane was equilibrated for 10 min in 0.1 M acetic acid and carbohydrates were oxidized for 20 min at room temperature in the dark with 10 mM sodium metaperiodate in 0.1 M acetic acid. The membrane was washed twice with 0.1 M acetic acid and once with 0.05% Tween-20, 0.1 M acetic acid. Biotin-hydrazide (Bio-Rad, USA) in 0.05% Tween-20 and 0.1 M acetic acid was then added and allowed to react for 60 min at RT in order to label the aldehydes that resulted from oxidation of the carbohydrates. After three washes with 0.05% Tween-20 in TBS, the membrane was blocked for 30 min and incubated with a 1:2,000 solution of streptavidin-alkaline phosphatase conjugate (Bio-Rad, USA). The membrane was washed again with TBST and stained with BCIP/NBT (Sigma, USA) as substrate.

Estimation of Glycoprotein Carbohydrate Content

The carbohydrate content of purified recombinant MSP1a was estimated using a glycoprotein carbohydrate estimation kit (Pierce, USA) according to the manufacturer's instructions.

Analysis of Monosaccharide Composition by Gas Chromatography

Analysis of the carbohydrate composition of pure MSP1a and MSP1b glycoproteins was performed using gas liquid chromatography of the trimethylsilyl glycoside derivatives (27). Affinity-purified MSP1a and MSP1b glycoproteins were dialyzed extensively against water and then freeze-dried. Inositol was added prior to drying the samples to serve as an internal standard. The protein samples were hydrolyzed with 1.5 M methanolic HCl and methyl acetate for 3 hours at 80° C. The samples were dried under a stream of N₂ and the sugars derivatized with a 3:1 trimethylsilyl:pyridine mixture for 15 minutes at room temperature. The trimethylsilyl sugar derivatives were dried, dissolved in isooctane and separated on a DB-1 fused silica capillary column (J & W Scientific Inc., USA) using a Varian 3300 gas chromatographer (Sunnyvale, USA). Monosaccharide amounts were calculated by relative comparison of the peak areas.

Analysis of Monosaccharide Composition by Capillary Electrophoresis

The monosaccharide composition of MSP1a and MSP1b was studied by capillary zone electrophoresis. Affinity-purified glycoproteins (2 μg) and 3-O-methyl glucose as internal standard were dried in a centrifugal vacuum evaporator. The glycans were hydrolyzed to monosaccharides with trifluoroacetic acid at 121° C. for 60 min. The monosaccharides were then derivatized with a fluorescent label by adding 3 mg/ml anthranilic acid, 4% sodium acetate, 2% borate in methanol, and the labeling reaction was allowed to proceed at 80° C. for 2 h. The methanol was evaporated and the samples were dissolved in water. Analytical separation of derivatized monosaccharides was performed in a Biofocus 2000 CZE instrument (Bio-Rad, USA) and detection was achieved by laser induced fluorescence. The amount of individual monosaccharides was estimated by comparison to the internal standard.

Enzymatic Deglycosylation

Affinity-purified recombinant MSP1a and MSP1b proteins were denatured with SDS and β-mercaptoethanol prior to the enzymatic deglycosylation reaction to increase the efficiency of deglycosylation. Enzymes used in this study included endoglycosidases PNGase F (N-glycosidase F) and O-glycosidase DS, specific for N-linked oligosaccharides and Gal(β-1,3) GalNAc(α1), respectively, and exoglycosidases GALase III (β1-4 galactosidase), HEXase I (β1-2,3,4,6 N-acetylhexosaminidase), NANase II (β2-3,6 neuraminidase), specific for β1-4 galactose, β-linked N-acetylglucosamine, and α2-3 and α2-6 N-acetylneuraminic acid residues, respectively. These enzymes were provided in the Enzymatic Deglycosylation Enhancement Kit (Bio-Rad, USA) and were used following the manufacturer's instructions.

Chemical Deglycosylation with TFMS

Purified recombinant MSP1a protein (500 μg) was dialyzed extensively against 0.1% trifluoroacetic acid and then freeze-dried. The MSP1a protein was deglycosylated by anhydrous trifluoromethanesulfonic (TFMS) acid treatment according to the instructions of the GlycoFree Deglycosylation Kit (Glyko Inc., USA). The TFMS acid cleaves protein-linked glycans non-selectively from the glycoprotein while leaving the primary structure of the protein intact (42).

MALDI-TOF Mass Spectrometry Analysis

To confirm that the MSP1a amino acids were not modified by the chemical deglycosylation with TFMS acid, native and deglycosylated MSP1a proteins were digested with trypsin and the proteolytic fragments analyzed by Matrix-Assisted Laser Desorption/Ionization Time-of-Flight (MALDI-TOF) mass spectrometry, which was performed using a Voyager DE PRO mass spectrometer (Applied Biosystems, USA) in the positive mode with reflectron, 20 kV accelerating voltage, 70% grid voltage with delayed extraction. Affinity-purified protein preparations were digested with Trypsin Gold (Promega, USA) and extracted following the manufacturer's instructions. The protein digest samples and α-cyano-4-hydroxycinnamic acid (a CHCA) matrix (in 50% acetonitrile, 0.3% trifluoroacetic acid) were spotted on the MALDI plate and allowed to dry at room temperature. External mass calibration was achieved using a mixture of peptide standards containing des-Arg¹-Bradykinin, Angiotensin I, Glu¹-Fibrinopeptide B and ACTH 1-17 (Sigma, USA) that was spotted next to the sample. Spectra from 250 laser shots were summed to obtain the accumulated spectrum. The peak lists generated from the analysis of native and deglycosylated MSP1a proteins were compared.

Tick Cell Binding Assay

The capacity of glycosylated and deglycosylated recombinant MSP1a to bind to tick cell extract was determined using a modification an in vitro binding assay that has been used in several studies to define MSP1a as an adhesin for tick gut and cultured tick cells (14-17). Cultured IDE8 tick cells were sonicated in 0.1% Triton X-100 and centrifuged at 12,000×g. Tick cell proteins (1 μg/well) were used for coating a 96-well plate for 3 h at 37° C. The plate was washed three times with TBST and blocked for 2 h at 37° C. with 2% skim milk. Serial 1:2 dilutions of native and deglycosylated pure recombinant MSP1a protein were added to the tick cell extract starting at 10 μg/well. Recombinant MSP1b was used as a negative control of binding. After incubating for 1 h at 37° C., the plate was washed with TBST and incubated with 1:1,000 anti-FLAG M2 monoclonal antibody (Sigma, USA) for 1 h at 37° C. The plate was washed and incubated with 1:2,000 goat anti-mouse IgG horseradish peroxidase conjugate (KPL, USA) for 1 h at 37° C. TMB in 0.05 M phosphate-citrate buffer, pH 5, containing 0.03% sodium perborate (Sigma, USA) was used for color development. The reaction was stopped with 2 N H₂SO₄ and the OD was read at 450 nm.

Protein Sequence Analysis and Prediction of Glycosylation Sites

The amino acid sequences of MSP1a and MSP1b proteins from several isolates of A. marginale were obtained from GenBank (Table 1). The amino acid composition and the predicted molecular weight for each isolate were determined by use of the statistical analysis of protein sequences algorithm (11), and the observed molecular masses were estimated from the electrophoretic mobility in SDS-PAGE.

Prediction of potential O-glycosylation sites in the MSP1a and MSP1b protein sequences was performed using the NetOGlyc 2.0 algorithm (24). Potential N-glycosylation sites were predicted by identifyin Asn-X-Ser/Thr sequences present in the MSP 1a and MSP1b amino acid sequences.

Results

Sequence Analysis and Prediction of Potential Glycosylation Sites

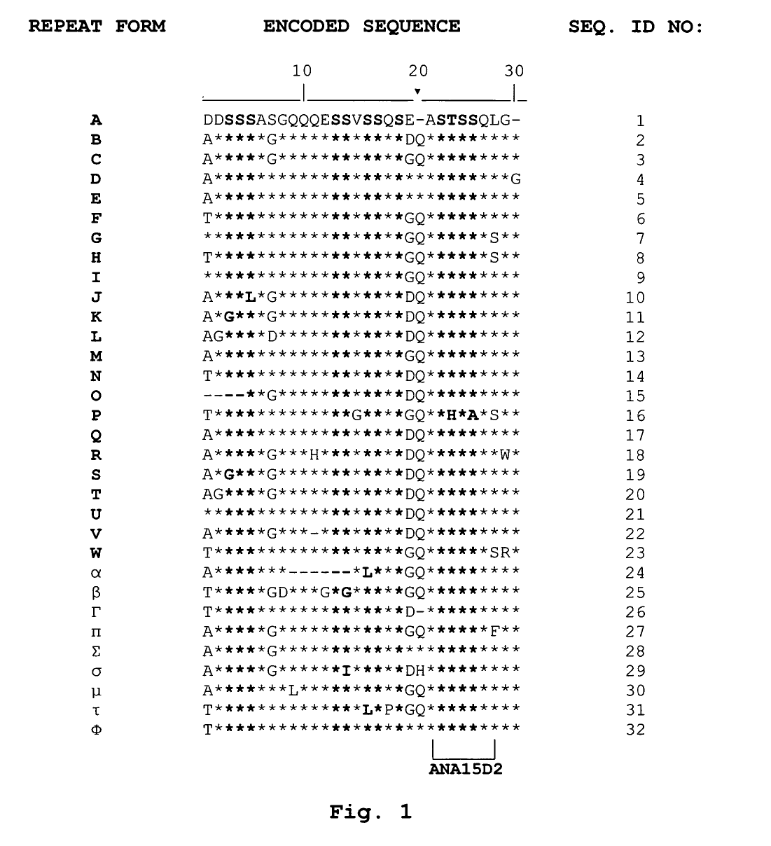

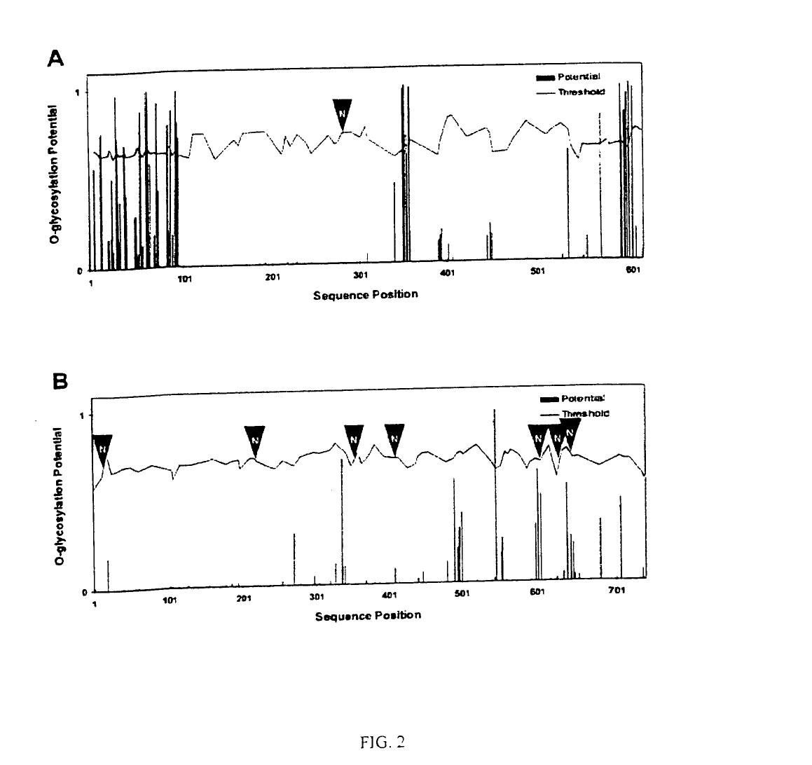

The amino acid sequences of MSP1a and MSP1b, deduced from the Oklahoma isolate msp1α and msplβ1 gene coding sequences, respectively, were analyzed for predicted N- and O-glycosylation sites, as well as for the amino acid composition. Oklahoma isolate MSP1a was found to be a Ser/Thr rich protein and contained 18% Ser/Thr (109 Ser+Thr/623 a.a.). The Ser/Thr content was particularly high in the region containing the tandemly repeated peptides (43%), suggesting an O linkage for possible carbohydrate modifications. Most of the Ser/Thr residues were conserved among the MSP1a repeats of different A. marginale isolates (FIG. 1). Although every Ser or Thr residue may be a potential O-glycosylation site, we used NetOGlyc 2.0 algorithm to predict which Ser/Thr residues were more likely to be glycosylated (FIG. 2). Of the 25 residues predicted to be O-glycosylated, 14 sites were identified in the N-terminal tandem repeats (FIG. 2A). Only one Asn-Xaa-Ser/Thr, as indicated by analysis of the potential N-glycosylation sites in the MSP1a sequence, was found to be present in the Oklahoma isolate MSP1a (FIG. 2A), and this Asn residue is not located in the repeated peptides.

MSP1b contained only 12% Ser/Thr (90 S+T/744 a.a.) and only one of these sites was predicted using NetOGly 2.0 to be O-glycosylated (FIG. 2B). However, seven Asn-Xaa-Ser/Thr sites were present in MSP1b (FIG. 2B), all of which may be potential N-glycosylation sites.

Molecular Weights of Native and Recombinant MSP1a and MSP1b Proteins

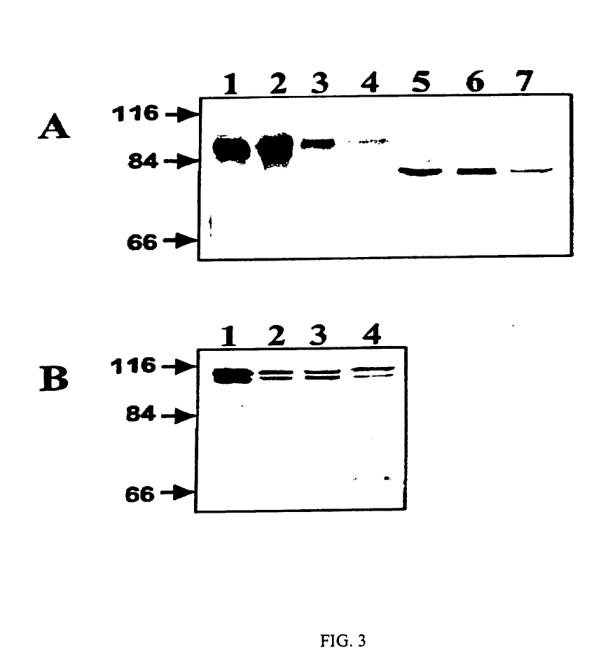

Although the molecular masses predicted from the deduced sequences of A. marginale MSP1a and MSP1b (Oklahoma isolate) were of 63 kDa and 79 kDa, respectively, the observed molecular weights of the recombinant E. coli-derived proteins were 90 kDa for MSP1a and 100 kDa for MSP1b (FIG. 3A, B). Native MSP1a and MSP1b proteins derived from A. marginale-infected cultured tick cells, erythrocytes and tick salivary glands had molecular weights similar to recombinant proteins (FIG. 3). The molecular weight of the MSP1a protein from a second isolate of A. marginale from Virginia, which contains a different number of tandemly repeated peptides, were also higher than predicted from their amino acid sequences (FIG. 3A, lanes 5-7). The recombinant MSP1a protein from the Virginia isolate (FIG. 3A, lane 5) had molecular weights similar to those of the native proteins (FIG. 3A, lanes 6, 7).

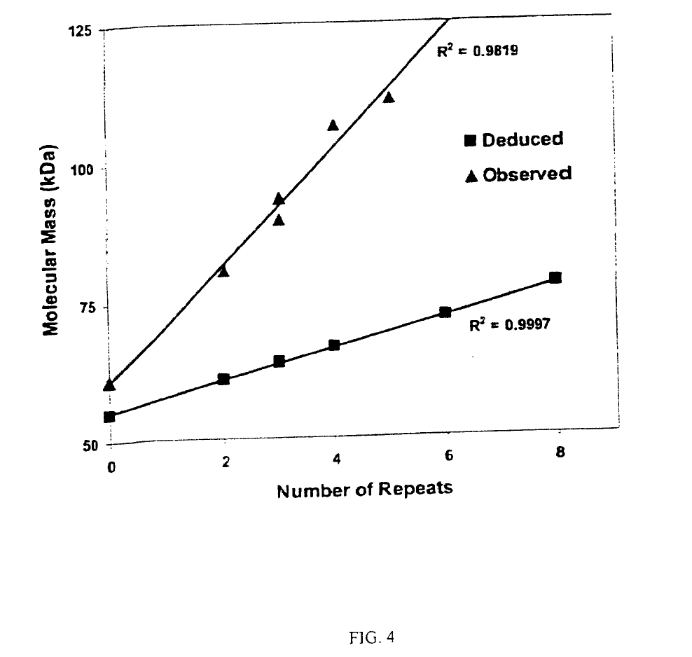

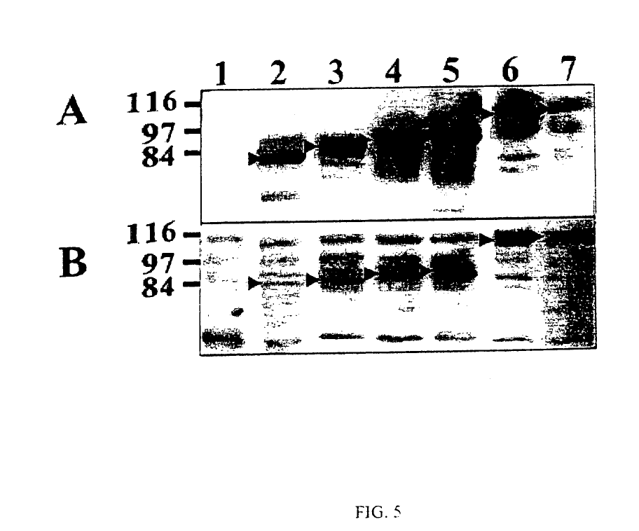

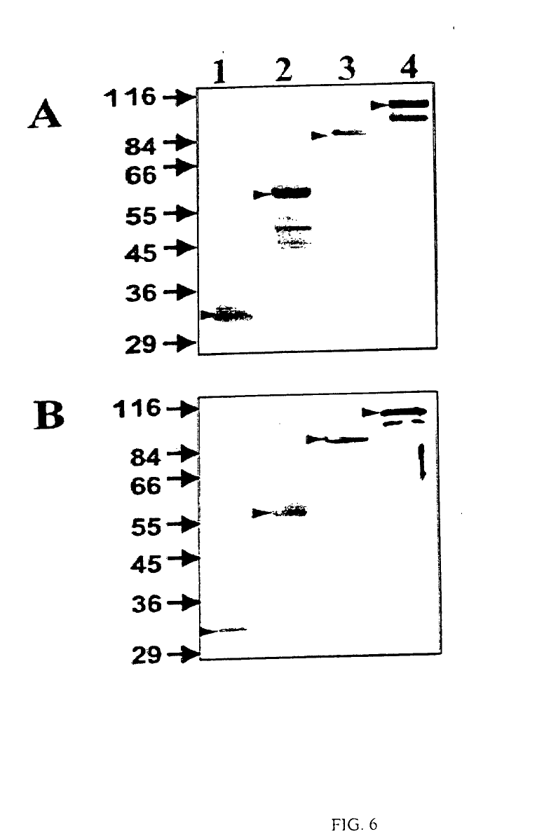

The msp1α gene that encodes for MSP1a was cloned from various geographic isolates of A. marginale and expressed in E. coli. The MSP1a proteins from these isolates contained 2-8 tandemly repeated peptides (Table 1). The deduced molecular masses of the proteins were calculated from their deduced primary sequence, and correlated with the number of repeated peptides in the same protein (FIG. 4). The correlation fit with the equation [MW(MSP1a)=2.8×N+55.5] in which N is the number of repeats and the intercept, 55.5 kDa, is an estimate of the molecular mass of the C-terminal region of MSP1a that is conserved among isolates. The slope, 2.8 kDa, represents the average deduced molecular mass of a single repeat. The observed molecular weights of the recombinant MSP1a proteins from all of the isolates studied, estimated from their electrophoretic mobility (FIG. 5A), were greater than the predicted molecular weights (FIG. 4), and the dependency with the number of repeats was described by the equation [MW(MSP1a)=10.5×N+62.5] which demonstrated that the molecular weights of both the conserved region and the repeated N-terminal peptides are greater than their deduced molecular masses. This equation also indicated that the average weight of a single repeat was 10.5 kDa, approximately 8 kDa greater than the molecular mass predicted from the amino acid sequence. The observed molecular weight of the MSP1a mutant that contained only the N-terminal repeats was approximately 30 kDa (FIG. 6, lane 1), similar to the molecular mass of 31.5 kDa predicted by the second equation, and was 3.6 times larger than the molecular mass predicted from the primary sequence and the first equation.

Detection of Glycosylation and Estimation of Carbohydrate Content

Glycosylation assays were performed in order to determine whether the difference between the deduced and observed molecular weights of MSP1a and MSP1b was due to glycosylation of the proteins. Crude extracts of recombinant E. Coli cells expressing the recombinant proteins were labeled with biotin-hydrazide after oxidation with sodium periodate. Glycosylation was detected in the recombinant MSP1a proteins from all the A. marginale isolates analyzed (FIG. 5B), as well as in the recombinant MSP1b protein (FIG. 6B, lane 4). The carbohydrate content was estimated to be 17% for MSP1a and >40% for MSP1b. Furthermore, glycosylation was detected on the two mutant MSP1a proteins expressed in E. Coli that contained either the conserved C-terminal region alone or the N-terminal repeats (FIG. 6B, lanes 1, 2).

Monosaccharide Compositional Analysis

The monosaccharide compositions of the recombinant MSP1a and MSP1b glycoproteins were determined by gas liquid chromatography. Four neutral sugars, glucose, galactose, mannose and xylose, were detected in the recombinant MSP1a (Table 2), while the recombinant MSP1b protein contained glucose, galactose and mannose (Table 2). Glucose was the most abundant monosaccharide in both recombinant proteins. These results were confirmed by capillary electrophoresis (data not shown).

Enzymatic Deglycosylation Analysis

The nature and structure of the glycans attached to MSP1a and MSP1b were characterized by treating affinity-purified recombinant MSP1a and MSP1b proteins with the endoglycosidases PNGase F, O-Glycosidase DS and the exoglycosidases GALase III, HEXase I and NANase II. Enzymatic treatment did not increase the electrophoretic mobility of MSP1a and MSP1b (data not shown). Therefore, these enzymes, which are specific for carbohydrate moieties commonly present in N- and O-glycoproteins, were not able to hydrolyze the glycans present in MSP1a and MSP1b glycoproteins.

Deglycosylation of MSP1a and Binding to Tick Cells

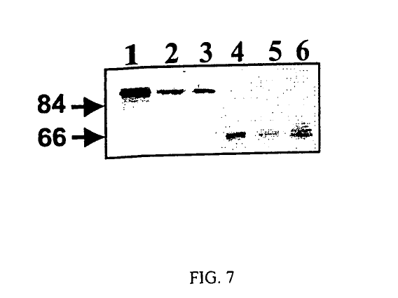

Recombinant MSP1a protein was chemically deglycosylated with TFMS acid in order to determine the role of carbohydrate modifications in the adhesive properties of the MSP1a for tick cells. Deglycosylation was determined by the increased electrophoretic mobility of the deglycosylated protein (FIG. 7). The peptide backbone of the MSP1a protein did not appear to be altered after acid deglycosylation treatment because the deglycosylated protein was recognized by three monoclonal antibodies specific for epitopes in the N-terminal repeats, the conserved C-terminal region and the C-terminally fused FLAG peptide (FIG. 7, lanes 4-6). Moreover, the peptide masses generated by the tryptic digestion of deglycosylated MSP1a, analyzed by MALDI-TOF mass spectrometry, matched peptide masses of the native MSP1a protein, indicating that covalent modifications were not introduced in the MSP1a amino acid backbone by the chemical deglycosylation with TFMS acid.

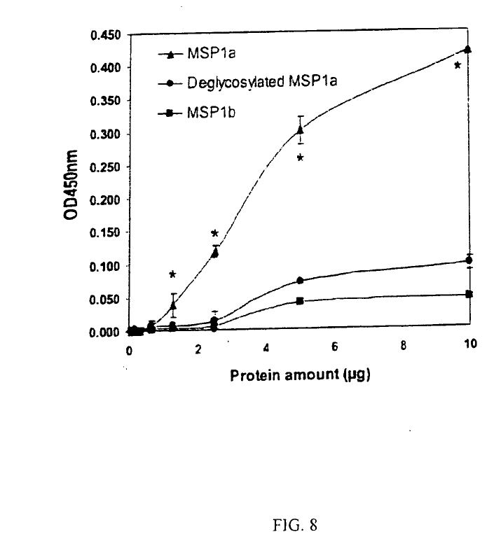

Tick cell binding assays were conducted to compare the adhesive properties of the native and deglycosylated MSP1a protein. The native MSP1a glycoprotein bound to tick cells (FIG. 8). Although the deglycosylated MSP1a protein also adhered to tick cells, its adhesive capacity was significantly reduced (P<0.01) with respect to native MSP1a.

Discussion

Several bacterial glycoproteins were reported recently and were shown to play a role in bacterial adhesion, invasion and pathogenesis. Glycosylation of outer membrane proteins was also described in several Gram-negative bacteria (5), including E. coli and the rickettsial tick-borne pathogens, E. canis and E. chaffeensis (30). In addition, recombinant proteins from A. phagocytophilum, E. chaffeensis and E. ruminantium expressed in E. coli were also found to be glycosylated (14).

Adhesion of A. marginale to host cells initiates the process of infection. Previous studies have demonstrated that polypeptides that compose the MSP1 complex, MSP1a and MSP1b, serve as A. marginale adhesins for tick cells and bovine erythrocytes (3, 15, 32). The functional domain of MSP1a was recently characterized and it was shown that the tandemly repeated peptides are necessary and sufficient to mediate adhesion of MSP1a to tick cells and bovine erythrocytes (17). A critical role of specific amino acids in the adhesive capacity of MSP1a was determined by use of a synthetic peptide model system (17).

The molecular weights of MSP1a and MSP1b have been determined by SDS-PAGE to be greater than the predicted molecular masses (1, 36), and the difference between the expected and observed molecular weights was posited to be due to the presence of carbohydrate modifications on these proteins (12, 39). In connection with the present invention it was discovered that both MSP1a and MSP1b from several A. marginale isolates are glycosylated. Glycosylation was particularly abundant in the N-terminal region of MSP1a that contains the repeated peptides. The repeated peptides of the Oklahoma isolate, which contain 43% Ser/Thr, were shown to be glycosylated and were predicted to be O-glycosylated using the NetOGlyc O-glycosylation prediction algorithm. Most of these Ser/Thr residues were found to be conserved among the different MSP1a repeats, particularly the residues at or next to the neutralization sensitive epitope and the amino acid in position 20 that appears to be important for adhesion to tick cells (1, 17, 39). Potential N-glycosylation sites were not present in this region, supporting the hypothesis that these glycans are O-linked. However, unusual modifications, known to occur in other bacterial glycoproteins (34), may also be present.

The number and type of potential glycosylation sites on MSP1a and MSP1b were different. While MSP1a contained a greater number of predicted O-glycosylation sites, MSP1b contained more potential N-glycosylation sites. Although only neutral sugars were detected in glycoproteins of both MSP1a and MSP1b, the difference in the number and type of glycosylation sites suggests that carbohydrate differences occur between the two proteins. The sugar composition of MSP1a and MSP1b indicates an unusual type of glycosylation in MSP1a and MSP1b. A similar carbohydrate composition has been described previously for the rickettsial recombinant proteins E. chaffeensis P120 and E. canis P140 expressed in E. coli (30). The absence of amino sugars was also consistent with previous studies in which MSP1a did not label with ³H-glucosamine (39). While N-acetylglucosamine and N-acetylgalactosamine are commonly present in the core carbohydrate structure of eukaryotic glycoproteins, the types of glycosylation identified in prokaryotes have been variable (5). Several lectins that recognize carbohydrate structures with N-acetylglucosamine and N-acetylgalactosamine did not bind to MSP1a (39), which provides further evidence of an unusual pattern of glycosylation. These results were also supported by the inability of exo- and endoglycosidases, specific for glycans that contain amino sugars, to deglycosylate recombinant MSP1a.

Although protein glycosylation in E. coli had been reported previously (5), glycosylation of heterologous recombinant proteins was thought not to occur until recently when a number of recombinant rickettsial proteins expressed in E. coli were shown to be glycosylated (14, 30). The ability of E. coli to glycosylate heterologous proteins appears to be specific for prokaryotic proteins that are glycosylated in their native form and therefore contain the required glycosylation sites. These recombinant proteins are also transported to the appropriate cellular location, most likely the plasma membrane, to become glycosylated.

Although we demonstrated previously that several recombinant rickettsial proteins expressed in E. coli were glycosylated (14), only two of these proteins, the A. marginale MSP1a and the E. ruminantium mucin-like protein, proved to be adherent for tick cells using an in vitro adhesion assay (14, 17). These two proteins had the highest content of Ser/Thr residues in the tandem repeats among those studied. These proteins appeared to be O-glycosylated and these O-linked glycans may be involved in adhesion to tick cells. In the present study, binding of recombinant MSP1a to tick cells was noticeably reduced when MSP1a was deglycosylated with TFMS acid, thus providing evidence that glycosylation plays a role in adhesion. While further studies are needed to confirm that the chemical deglycosylation did not introduce chemical modifications in amino acid residues of the protein that may have reduced the adhesive properties of the protein, it should be noted that chemical deglycosylation of other proteins did not affect their biological activity (42).

It has further been demonstrated that the deglycosylated peptide backbone of MSP1a was able to bind to tick cell extracts, although at reduced levels. The combined results of these and previous studies where synthetic peptides were used that model the MSP1a repeats (17) suggest that both the MSP1a peptidic backbone and its carbohydrate modifications are involved in the cooperative interaction with putative host cell receptors. Recent studies on a closely-related organism, A. phagocytophilum, demonstrated that A. phagocytophilum binds cooperatively to sites on the N-terminal peptide of human PSGL-1 and to carbohydrate moieties on the same or different molecules (26, 44). In addition to MSP1a, MSP1b and MSP2 have been shown to be adhesins for bovine erythrocytes (3, 15, 32) and, therefore, these MSPs may cooperate in adhesion of A. marginale to erythrocytes.

Glycosylation of A. marginale surface proteins may also influence the capacity of the pathogen to generate antigenic diversity and to escape the host's immune response, as has been demonstrated for other bacterial and viral pathogens (2, 43). While major amino acid changes may affect the conformation and thus function of the protein, minor amino acid changes may only alter the pattern of glycosylation, thus generating new antigenic variants that may allow pathogens to evade the host immune response (43). In addition, glycosylation of proteins can occur in multiple forms, a phenomenon known as microheterogeneity, which may further contribute to antigenic diversity. Completion of the sequence of the A. marginale genome may provide the opportunity to identify genes encoding for the glycosylation machinery, as well as other glycosylated proteins. This approach has shown to be productive for the study of other pathogenic bacteria (40).

This research in connection with the present invention provides the first evidence of the role of glycosylation of A. marginale surface proteins in adhesion to host cells and may contribute to development of more effective vaccine strategies for control of this economically important pathogen of cattle.

The preparation of pharmaceutical compositions (vaccines) utilizing the aforedescribed glycans as distinct antigenic components is easily accomplished using well known methods and techniques. The vaccine and/or antigen preparation is combined into a formulation in an amount effective to provide for a protective immune response against infection with A. marginale. A protective immune response against A. marginale decreases the clinical signs of anaplasmosis. Clinical symptoms of anaplasmosis include a reduction in packed red cell volume of about 25 to 80% and parasitemia of the red blood cells of about 15 to 70%. A decrease in the symptoms of anaplasmosis includes prevention of the reduction in the packed red cell volume and a decrease in percent parasitemia. Preferably, a protective response includes packed red cell volume change of 25% or less compared with control animals and/or a decrease in parasitemia to about 5 to 25% of the red blood cells or less depending on the conditions. Measurements of packed red cell volume and percent parasitemia are conducted using standard methods. Vaccine preparations are combined with physiologically acceptable carriers to form vaccines. The preferred physiologically acceptable carrier is an oil-based adjuvant.

Preferably, the inventive formulation is set to contain about 100 micrograms of antigen in an oil-based adjuvant such as Xtend® III (Grand Laboratories, Larchwood, Iowa).

The vaccines may be administered by a variety of routes including intravenously, intraperitoneally, intramuscularly, and subcutaneously. The preferred route of administration is subcutaneous. The vaccine can be administered in a single dose or multiple doses until the desired effect is achieved.

While the invention has been described with a certain degree of particularity, it is understood that the invention is not limited to the embodiment(s) set for herein for purposes of exemplification, but is to be limited only by the scope of the attached claim or claims, including the full range of equivalency to which each element thereof is entitled.

References, all of which are incorporated herein by reference.
1. Allred, D. R., T. C. McGuire, G. H. Palmer, S. R. Leib, T. M. Harkins, T. F. McElwain, and A. F. Barbet. 1990. Molecular basis for surface antigen size polymorphisms and conservation of a neutralization-sensitive epitope in Anaplasina marginale. Proc. Natl. Acad. Sci. 87:3220-3224.
2. Alm, R. A., P. Guerry, M. F. Power, and T. J. Trust. 1992. Variation in antigenicity and molecular weight of Campylobacter coli VC167 flagellin in different genetic backgrounds. J. Bacteriol. 174:4230-4238.
3. Barbet, A. F., and D. R. Allred. 1991. The msp1 beta multigene family of Anaplasma marginale: nucleotide sequence analysis of an expressed copy. Infect. Immun. 59:971-976.
4. Barbet, A. F., G. H. Palmer, P. J. Myler, and T. C. McGuire. 1987. Characterization of an immunoprotective protein complex of Anaplasma marginale by cloning and expression of the gene coding for polypeptide Am105L. Infect. Immun. 55:2428-2435.
5. Benz, I., and M. A. Schmidt. 2002. Never say never again: protein glycosylation in pathogenic bacteria. Mol. Microbiol. 45:267-276.
6. Blouin, E. F., A. F. Barbet, Jooyoung Yi, K. M. Kocan, and J, T. Saliki. 2000. Establishment and characterization of an Oklahoma isolate of Anaplasma marginale in cultured Ixodes scapularis cells. Vet. Parasitol. 87:301-313.
7. Blouin, E. F., and K. M. Kocan. 1998. Morphology and development of Anaplasma marginale (Rickettsiales: Anaplasmataceae) in cultured Ixodes scapularis (Acari:Ixodidae) cells. J. Med. Entomol. 35:788-797.
8. Blouin, E. F., J. T. Saliki, J. de la Fuente, J. C. Garcia-Garcia, and K. M. Kocan. 2003. Antibodies to Anaplasma marginale major surface proteins 1a and 1b inhibit infection for cultured tick cells. Vet. Parasitol. 111:247-260.
9. Bouchez-Mahiout, I., C. Doyen, and M. Lauriere. 1999. Accurate detection of both glycoproteins and total proteins on blots: control of side reactions occurring after periodate oxidation of proteins. Electrophoresis 20:1412-1417.
10. Bowie, M. V., J. de la Fuente, K. M. Kocan, E. F. Blouin, and A. F. Barbet. 2000. Conservation of major surface protein 1 genes of the ehrlichial pathogen Anaplasma marginale during cyclic transmission between ticks and cattle. Gene 282:95-102.
11. Brendel, V., P. Bucher, I. Nourbakhsh, B. E. Blaisdell, and S. Karlin. 1992. Methods and algorithms for statistical analysis of protein sequences. Proc. Natl. Acad. Sci. USA 89:2002-2006.
12. Brown, W. C., G. H. Palmer, H. A. Lewin, and T. C. McGuire. 2001. CD4(+) T lymphocytes from calves immunized with Anaplasma marginale major surface protein 1 (MSP1), a heteromeric complex of MSP1a and MSP1b, preferentially recognize the MSP1a carboxyl terminus that is conserved among strains. Infect. Immun. 69:6853-6862.
13. Camacho-Nuez, M., M. de Lourdes Munoz, C. E. Suarez, T. C. McGuire, W. C, Brown, and G. H. Palmer. 2000. Expression of polymorphic msplβ genes during acute Anaplasma marginale rickettsemia. Infect. Immun. 68:1946-1952.
14. de la Fuente, J., J. C. Garcia-Garcia, A. F. Barbet, E. F. Blouin, and K. M. Kocan. 2003. Adhesion of outer membrane proteins containing tandem repeats of Anaplasma and Ehrlichia species (Rickettsiales: Anaplasmataceae) to tick cells. Vet. Microbiol., in press.
15. de la Fuente, J., J. C. Garcia-Garcia, E. F. Blouin, and K. M. Kocan. 2001. Differential adhesion of major surface proteins 1a and 1b of the ehrlichial cattle pathogen Anaplasma marginale to bovine erythrocytes and tick cells. Int. J. Parasitol. 31:145-i 53.
16. de la Fuente, J., J. C. Garcia-Garcia, E. F. Blouin, and K, M. Kocan. 2001. Major surface protein 1a effects tick infection and transmission of the ehrlichial pathogen Anaplasma rnarginale. Int. J. Parasitol. 31:1705-1714.
17. de la Fuente, J., J. C. Garcia-Garcia, E. F. Blouin, and K. M. Kocan. 2003. Characterization of the functional domain of major surface protein 1a involved in adhesion of the rickettsia Anaplasma marginale to host cells. Vet. Microbiol. 91:265-283.
18. de la Fuente, J., J. C. Garcia-Garcia, E. F. Blouin, S. D. Rodriguez, M. A. Garcia, and K. M. Kocan. 2002. Evolution and function of tandem repeats in the major surface protein 1a of the ehrlichial pathogen Anaplasma marginale. Anim. Health Res. Rev. 2:163-173.
19. de la Fuente, J., J. C. Garcia-Garcia, E. F. Blouin, J. T. Saliki, and K. M. Kocan. 2001. Infection of tick cells and bovine erythrocytes with one genotype of the intracellular ehrlichia Anaplasma marginale excludes infection with other genotypes. Clin. Diagn. Lab. Imniunol. 9:658-668.
20. de la Fuente, J., R. A. Van Den Bussche, and K. M. Kocan. 2001. Molecular phylogeny and biogeography of North American isolates of Anaplasma marginale (Rickettsiaceae: Ehrlichieae). Vet. Parasitol. 97:65-76.
21. Dikmans, G. 1950. The transmission of anaplasmosis. Am. J. Vet. Res. 11:5-16.
22. Eriks, I. S., D. Stiller, W. L. Goff, M. Panton, S. M. Parish, T. F. McElwain, and G. H. Palmer. 1994. Molecular and biological characterization of a newly isolated Anaplasma marginale strain. J. Vet. Diagn. Invest. 6:435-441.
23. Ewing, S. A. 1981. Transmission of Anaplasma marginale by arthropods, p. 395-423. In Proc. 7 National Anaplasmosis Conference, Mississippi State University, Miss.
24. Hansen, J. E., O. Lund, N. Tolstrup, A. A. Gooley, K. L. Williams, and S. Brunak. 1998. NetOglyc: prediction of mucin type O-glycosylation sites based on sequence context and surface accessibility. Glycoconjugate J. 15:115-130.
25. Haselbeck, A., and W. Hösel. 1993. Immunological detection of glycoproteins on blots based on labeling with digoxigenin. Methods Mol. Biol. 14:161-73,
26. Herron, M. J., C. M. Nelson, J. Larson, K. R. Snapp, G. S. Kansas, and J. L. Goodman. 2000. Intracellular parasitism by the human granulocytic ehrlichiosis bacterium through the P-selectin ligand, PSGL-1. Science 288:1653-1656.
27. Komalavilas, P., and A. J. Mort. 1989. The acetylation of O-3 of galacturonic acid in the rhamnose-rich portion of pectins. Carbohyd. Res. 189:261-272.
28. Kuttler, K. L. 1984. Anaplasma infections in wild and domestic ruminants: a review. J. Wild. Dis. 20:12-20.
29. Laemmli, U. K. 1970. Cleavage of structural proteins during the assembly of the head of bacteriophage T4. Nature 227:680-685.
30. McBride, J. W., X. J. Yu, and D. H. Walker. 2000. Glycosylation of homologous immunodominant proteins of Ehrlichia chaffeensis and Ehrlichia canis. Infect. Immun. 68: 13-18.
31. McGarey, D. J., and P. R. Allred. 1994. Characterization of hemagglutinating components on the Anaplasma marginale initial body surface and identification of possible adhesins. Infect. Immun. 62:4587-4593.
32. McGarey, D. J., A, F. Barbet, G. H. Palmer, T. C. McGuire, and P. R. Allred. 1994. Putative adhesins of Anaplasma marginale: major surface polypeptides 1a and 1b. Infect. Immun. 62:4594-4601.
33. McGuire, T. C., G. H. Palmer, W. L. Goff, M. I. Johnson, and W. C. Davis. 1984. Common and isolate-restricted antigens of Anaplasma marginale detected with monoclonal antibodies. Infect. Immun. 45:697-700.
34. Messner, P., R. Christian, C. Neuninger, and G. Schulz. 1995. Similarity of “core” structures in two different glycans of tyrosine-linked eubacterial S-layer glycoproteins. J. Bacteriol. 177:2188-2193.
35. Munderloh, U. G., E. F. Blouin, K. M. Kocan, N. L. Ge, W. L. Edwards, and T. J. Kurtti. 1996. Establishment of the tick (Acari: Ixodidae)-bome cattle pathogen Anaplasma marginale (Rickettsiales: Anaplasmataceae) in tick cell culture. J. Med. Bitt. 33:656-664.
36. Oberle, S. M., G. H. Palmer, A. F. Barbet, and T. C. McGuire. 1988. Molecular size variations in an immunoprotective protein complex among isolates of Anaplasma marginale. Infect. Immun. 56:1567-1573.
37. Palmer, G. H., A. F. Barbet, G. H. Cantor, and T. C. McGuire. 1989. Immunization of cattle with the MSP-1 surface protein complex induces protection against a structurally variant Anaplasma marginale isolate. Infect. Immun. 57:3666-3669.
38. Palmer, G. H., F. R. Rurangirwa, and T. F. McElwain. 2001. Strain composition of the ehrlichia Anaplasma marginale within persistently infected cattle, a mammalian reservoir for tick transmission. J. Clin. Microbiol. 39:631-635.
39. Palmer, G. H., S. D. Waghela, A. F. Barbet, W. C. Davis, and T. C. McGuire. 1987. Characterization of a neutralization sensitive epitope on the AM 105 surface protein of Anaplasma marginale. Int. J. Parasitol. 17:1279-1285.
40. Power, P. M., and M. P. Jennings. 2003. The genetics of glycosylation in Gram-negative bacteria. FEMS Microbiol. Lett. 218:211-222.
41. Ristic, M., and A. M. Watrach. 1963. Anaplasmosis. VI. Studies and a hypothesis concerning the cycle of development of the causative agent. Am. J. Vet. Res. 24:267-276.
42. Tams, J. W., and K. G. Welinder. 1995. Mild chemical deglycosylation of horseradish peroxidase yields a fully active, homogeneous enzyme. Anal. Biochem. 228:48-55.
43. Wei, X., J. M. Decker, S. Wang, H. Hui, J. C. Kappes, X. Wu, J. F. Salazar-Gonzalez, M. G. Salazar, J. M. Kilby, M. S. Saag, N. L. Komarova, M. A. Nowak, B. H. Hahn, P. D. Kwong, and C. M. Shaw. 2003. Antibody neutralization and escape by HIV-1. Nature 422:307-3 12.

44. Yago, T., A. Leppanen, J. A. Carlyon, M. Akkoyunlu, S. Karmakar, E. Fikrig, R. D. Cummings, and R. P. McEver. 2003. Structurally distinct requirements for binding of P-selectin glycoprotein ligand-1 and sialyl Lewis x to Anaplasma phagocytophilum and P-selectin. J. Biol. Chem. 278:37987-37997.

TABLE 1
Anaplasma marginale isolates and MSP1a
proteins included in the study.

| | MSP1a | | GenBank | |
|---|---|---|---|---|
| Isolate | No. | Predicted | Accession | |
| Name/Origin | Repeats | Mol. Wt^{a} | No. | Reference |
| Virginia | 2 | 60.8 | M32870 | McGuire et al. (33) |
| Oklahoma | 3 | 63.5 | AY010247 | Blouin et al. (6) |
| California | 3 | NR | AY010242 | de la Fuente et al. |
| | | | | (20) |
| St. Maries, | 3 | 63.5 | AF293062 | Eriks et al. (22) |
| ID | | | | |
| Rasmussen | 3 | 63.5 | AF293064 | Palmer et al. (38) |
| South Dakota | 3 | 63.7 | AF293063 | Palmer et al. (38) |
| Texas | 4 | NR | AF428091 | McGuire et al. (33) |
| Washington | 4 | 66.4 | M32869 | Allred et al. (1) |
| Okeechobee, | 5 | NR | AY010244 | de la Fuente et al. |
| FL | | | | (16) |
| Idaho | 6 | 71.7 | M32868 | Allred et al. (1) |
| Florida | 8 | 77.5 | M32871 | Allred et al. (1) |

^{a}The molecular mass of the MSP1a proteins was predicted from their amino acid sequences using the statistical analysis of protein sequences algorithm. NR indicates MSP1a proteins for which the complete coding sequence have not been reported.

TABLE 2
Monosaccharide composition of recombinant
Anaplasma marginale MSP1a and MSP1b^{a}.

| Monosaccharide | MSP1a | MSP1b |
|---|---|---|
| Glucose | 66.5 | 67.3 |
| Galactose | 16.0 | 12.1 |
| Mannose | 6.0 | 20.6 |
| Xylose | 11.5 | 0.0 |

^{a}Amounts of monosaccharides are expressed as the percent of total monosaccharides in the glycoprotein, as determined by gas chromatography of the trimethylsilyl glycoside derivatives.

1. A polypeptide comprising an isolated and purified glycan derived from Anaplasma marginale major surface protein (MSP)1.

2. The polypeptide according to claim 1 wherein said glycan is derived from the N-terminal repeated peptides of MSP1a.

3. A pharmaceutical composition comprising at least one isolated and purified glycan derived Anaplasma marginale major surface protein (MSP)1, wherein said composition further comprises a pharmaceutically acceptable carrier or diluent.

4. The composition according to claim 1 wherein said glycan is derived from the N-terminal repeated peptides of MSP1a.

5. A method for inducing or enhancing an immune response in a ruminate against infection by A. marginale, said method comprising administering to said ruminant an effective amount of the pharmaceutical composition of claim 3.

6. The method according to claim 5, wherein said glycan is derived from the N-terminal repeated peptides of MSP1a.


SEQUENCE LISTING (32 sequences)

SEQ ID NO: 1 - protein, 28 aa, Anaplasma marginale
DDSSSASGQQQESSVSSQSEASTSSQLG

SEQ ID NO: 2 - protein, 29 aa, Anaplasma marginale
ADSSSAGGQQQESSVSSQSDQASTSSQLG

SEQ ID NO: 3 - protein, 29 aa, Anaplasma marginale
ADSSSAGGQQQESSVSSQSGQASTSSQLG

SEQ ID NO: 4 - protein, 29 aa, Anaplasma marginale
ADSSSASGQQQESSVSSQSEASTSSQLGG

SEQ ID NO: 5 - protein, 28 aa, Anaplasma marginale
ADSSSASGQQQESSVSSQSEASTSSQLG

SEQ ID NO: 6 - protein, 29 aa, Anaplasma marginale
TDSSSASGQQQESSVSSQSGQASTSSQLG

SEQ ID NO: 7 - protein, 29 aa, Anaplasma marginale
DDSSSASGQQQESSVSSQSGQASTSSQSG

SEQ ID NO: 8 - protein, 29 aa, Anaplasma marginale
TDSSSASGQQQESSVSSQSGQASTSSQSG

SEQ ID NO: 9 - protein, 29 aa, Anaplasma marginale
DDSSSASGQQQESSVSSQSGQASTSSQLG

SEQ ID NO: 10 - protein, 29 aa, Anaplasma marginale
ADSSLAGGQQQESSVSSQSDQASTSSQLG

SEQ ID NO: 11 - protein, 29 aa, Anaplasma marginale
ADGSSAGGQQQESSVSSQSDQASTSSQLG

SEQ ID NO: 12 - protein, 29 aa, Anaplasma marginale
AGSSSADGQQQESSVSSQSDQASTSSQLG

SEQ ID NO: 13 - protein, 29 aa, Anaplasma marginale
ADSSSASGQQQESSVSSQSGQASTSSQLG

SEQ ID NO: 14 - protein, 29 aa, Anaplasma marginale
TDSSSASGQQQESSVSSQSDQASTSSQLG

SEQ ID NO: 15 - protein, 25 aa, Anaplasma marginale
SAGGQQQESSVSSQSDQASTSSQLG

SEQ ID NO: 16 - protein, 29 aa, Anaplasma marginale
TDSSSASGQQQESSGSSQSGQASHSAQSG

SEQ ID NO: 17 - protein, 29 aa, Anaplasma marginale
ADSSSASGQQQESSVSSQSDQASTSSQLG

SEQ ID NO: 18 - protein, 29 aa, Anaplasma marginale
ADSSSAGGQQHESSVSSQSDQASTSSQLW

SEQ ID NO: 19 - protein, 29 aa, Anaplasma marginale
ADGSSAGGQQQESSVSSQSDQASTSSQLG

SEQ ID NO: 20 - protein, 29 aa, Anaplasma marginale
AGSSSAGGQQQESSVSSQSDQASTSSQLG

SEQ ID NO: 21 - protein, 29 aa, Anaplasma marginale
DDSSSASGQQQESSVSSQSDQASTSSQLG

SEQ ID NO: 22 - protein, 28 aa, Anaplasma marginale
ADSSSAGGQQESSVSSQSDQASTSSQLG

SEQ ID NO: 23 - protein, 29 aa, Anaplasma marginale
TDSSSASGQQQESSVSSQSGQASTSSQSR

SEQ ID NO: 24 - protein, 23 aa, Anaplasma marginale
ADSSSASGVLSQSGQASTSSQLG

SEQ ID NO: 25 - protein, 29 aa, Anaplasma marginale
TDSSSAGDQQQGSGVSSQSGQASTSSQLG

SEQ ID NO: 26 - protein, 28 aa, Anaplasma marginale
TDSSSASGQQQESSVSSQSDASTSSQLG

SEQ ID NO: 27 - protein, 29 aa, Anaplasma marginale
ADSSSAGGQQQESSVSSQSGQASTSSQFG

SEQ ID NO: 28 - protein, 28 aa, Anaplasma marginale
ADSSSAGGQQQESSVSSQSEASTSSQLG

SEQ ID NO: 29 - protein, 29 aa, Anaplasma marginale
ADSSSAGGQQQESIVSSQSDHASTSSQLG

SEQ ID NO: 30 - protein, 29 aa, Anaplasma marginale
ADSSSASGLQQESSVSSQSGQASTSSQLG

SEQ ID NO: 31 - protein, 29 aa, Anaplasma marginale
TDSSSASGQQQESSVLSPSGQASTSSQLG

SEQ ID NO: 32 - protein, 28 aa, Anaplasma marginale
TDSSSASGQQQESSVSSQSEASTSSQLG